(19) United States
(12) Patent Application Publication
(10) Pub. No.: US 2020/0085324 A1
(43) Pub. Date: Mar. 19, 2020

(54) METHOD FOR ESTABLISHING AT LEAST ONE BLOOD PRESSURE VALUE OF A TEST SUBJECT

(71) Applicants: HUMAN RESEARCH INSTITUT FÜR GESUNDHEITSTECHNOLOGIE UND PRÄVENTIONSFORSCHUNG GMBH, Weiz (AT); JOYSYS GMBH, Weiz (AT); AMS AG, Premstätten (AT)

(72) Inventors: Maximilian MOSER, Keutschach (AT); Thomas HASSLER, Graz (AT); Thomas STOCKMEIER, Premstätten (AT); Bernhard GRUBER, Gratwein (AT)

(73) Assignees: HUMAN RESEARCH INSTITUT FÜR GESUNDHEITSTECHNOLOGIE UND PRÄVENTIONSFORSCHUNG GMBH, Weiz (AT); JOYSYS GMBH, Weiz (AT); AMS AG, Premstätten (AT)

(21) Appl. No.: 16/616,015

(22) PCT Filed: May 22, 2018
(86) PCT No.: PCT/EP2018/063406

§ 371 Date: Nov. 22, 2019

(30) Foreign Application Priority Data
May 22, 2017 (AT) A 50434/2017

(51) Int. Cl. A61B 5/021; A61B 5/0402; A61B 5/00; A61B 5/024

(52) U.S. Cl. CPC A61B 5/02125; A61B 5/02405; A61B 5/4035; A61B 5/0402

(57) ABSTRACT

The invention relates to a method for determining at least one blood pressure value of a subject. According to the invention, a heartbeat signal of a subject, in particular an ECG, is determined by means of a heartbeat measuring arrangement and transmitted to a circulation parameter determination unit, wherein the circulation parameter determination unit determines at least one value of an autonomic tone, in particular a vagal tone and/or a heart rate variability and/or a sympathetic tone and/or an autonomic quotient, from a determined time profile of the heartbeat signal, wherein the circulation parameter determination unit determines the at least one blood pressure value taking into account the at least one value of the autonomic tone, and the determined blood pressure value is output.

### CROSS-REFERENCE TO RELATED APPLICATIONS

The present application is a national phase application of PCT Application No. PCT/EP2018/063406, filed May 22, 2018, entitled “METHOD FOR DETERMINING AT LEAST ONE BLOOD PRESSURE VALUE OF A SUBJECT”, which claims the benefit of Austrian Patent Application No. A 5034/2017, filed May 22, 2017, each of which is incorporated by reference in its entirety.

### BACKGROUND OF THE INVENTION

### 1. Field of the Invention

The invention relates to a method for determining at least one blood pressure value of a subject.

### 2. Description of the Related Art

Blood pressure, as one of the most essential blood circulation parameters, is classically measured by means of a cuff on the upper arm. Although this is a well-known method of measurement, it places certain demands on the person making the measurement, and is hardly feasible for non-professionals.

In addition, other methods for determining blood pressure are known; in these the duration of a pulse wave from the heart to a peripheral blood vessel is measured, and a conclusion about blood pressure is made therefrom. A disadvantage of such methods and/or devices known to-date is the proven low accuracy of the blood pressure values determined in these ways. Control measurements have shown that the variations in such “measurements” with known devices is so inaccurate that they are meaningless and/or useless from a medical point of view.

The object of the invention is therefore to provide a method of the type mentioned above, by means of which the mentioned disadvantages can be avoided, and by means of which it is possible to accurately determine at least one blood pressure value, in particular a chronological profile of blood pressure values, of a subject in a simple manner and with few measured values.

### SUMMARY OF THE INVENTION

According to the invention, this is achieved by a method for determining at least one blood pressure value of a test subject, wherein a heartbeat signal, in particular an ECG, of a subject is determined by means of a heartbeat measuring arrangement and transmitted to a circulation parameter determination unit. The circulation parameter determination unit determines at least one value of an autonomic tone, in particular a vagal tone and/or a heart rate variability and/or a sympathetic tone and/or an autonomic quotient, from a determined time profile of the heartbeat signal. The circulation parameter determination unit determines the at least one blood pressure value taking into account the at least one value of the autonomic tone, and the determined blood pressure value is output.

This approach enables an accurate determination of at least one blood pressure value of a subject in a simple manner Accuracy has been significantly improved by additionally taking autonomic tone into account. It has been shown that autonomic tone has a considerable influence on the blood pressure profile. In particular, vagal tone and/or heart rate variability and/or sympathetic tone and/or autonomic quotient are used and/or considered as autonomic tone, wherein another tone controlled by the brain stem may also be taken into account. Autonomic quotients are known from the applicant's filing AT 517 071 B1. Autonomic tone has a direct influence on the peripheral resistance of blood vessels and thus on actual blood pressure.

By means of the present measuring method, blood pressure can be determined by means of fewer measured values and/or within the time of much fewer heart beats. With this approach, the heartbeat to heartbeat rhythm of the blood pressure can also be measured. Therefore, in contrast to the conventional method of measurement by means of a cuff, a blood pressure profile for each individual heartbeat can be determined—as well as a blood pressure rhythm.

Furthermore, a value for arterial elasticity can also be determined from the autonomic tone and/or the pulse wave velocity alone.

The invention further relates to a device for determining at least one circulation parameter of a subject, the device comprising a heartbeat measuring arrangement, a pulse measuring arrangement and a circulation parameter determination unit, characterized in that the device is designed to carry out a method according to any of the exemplary methods described herein.

The object of the invention is therefore to provide a device of the type mentioned above, by means of which the noted disadvantages can be avoided, and by means of which it is possible in a simple manner to accurately determine at least one circulation parameter of a subject—in particular, blood pressure.

This is achieved according to the invention by the features discussed above, which enable the advantages claimed above for the method.

The dependent claims relate to further advantageous embodiments of the invention.

The wording of the claims is hereby expressly referenced, and the claims shall be considered incorporated into the description at this point and reproduced verbatim by reference.

### BRIEF DESCRIPTION OF THE DRAWINGS

The invention will be described in more detail with reference to the accompanying drawings, in which only preferred embodiments are shown by way of example. In the drawings:

FIG. 1 shows a block diagram of a preferred embodiment of a method according to the invention;

FIG. 2 shows a block diagram of a preferred embodiment of a device according to the invention;

FIG. 3 shows a preferred embodiment of the contact area of a device according to the invention, as a toilet seat; and

FIG. 4 shows a preferred embodiment of a device according to the invention as a scale.

### DETAILED DESCRIPTION

In a method for determining at least one blood pressure value of a subject, a heartbeat signal of the subject is detected by means of a heartbeat measuring arrangement 2. This may be any type of a corresponding measuring arrangement, wherein an ECG unit is particularly preferred. Furthermore, ballistocardiography, ultrasound cardiography, magnetocardiography, sonocardiography, or capacitive measurements can also be used for recording the heartbeat signal, and the heartbeat measuring arrangement 2 can also be designed accordingly.

The determined heartbeat signal is transmitted to a circulation parameter determination unit 4, which preferably comprises a microcomputer and/or microcontroller. The circulation parameter determination unit 4 determines at least one value of an autonomic tone from the chronological profile of the heartbeat signal. Preference is given to vagal tone and/or heart rate variability and/or sympathetic tone and/or an autonomic quotient. The determination takes place during evaluation of an ECG in a manner known per se, by recording the different durations of successive R-R intervals. R denotes the R-wave in a manner known per se. In this regard, reference is further made to the applicant's AT 515 102 and WO 2015/176088, in which the measurement of vagal tone is described in detail.

The circulation parameter determination unit 4 subsequently determines a value for a blood pressure of the subject and outputs this value via a display, and/or stores or transmits this value. The value for the blood pressure is preferably continuously determined and/or updated. Furthermore, values for the systolic and the diastolic blood pressure, and/or the blood pressure amplitude, are preferably determined and output.

The circulation parameter determination unit 4 preferably also determines values for arterial elasticity and/or a pulse wave velocity PWV, and outputs and/or stores these.

In determining the blood pressure value, the at least one value of the autonomic tone, in particular the heart rate variability, is taken into account.

A first preferred implementation of the method according to the invention will be described below.

The circulation parameter determination unit (4) determines from the autonomic tone a time value of a pre-ejection period PEP. Via this pre-ejection period PEP, autonomic tone is taken into account in determining the at least one blood pressure value. It has proven to be advantageous if a statistically meaningful value is used, rather than just any value of an autonomic tone.

A first preferred equation for the median of the measured vagal tone values is:

VT=median of {log[absolute(RRₙ₊₁−RRₙ)]}

A second preferred equation for the mean of the measured vagal tone values is:

$$VT = log \vert \frac{1}{N} {\sum }_{i = 0}^{N} [ absolute ( {RR}_{n + 1} - {RR}_{n} ) ] \vert$$

It has proven to be advantageous if n is greater than or equal to 4.

Furthermore, it has proven advantageous for the accuracy of the determined blood pressure value if these values VT are further corrected and/or normalized.

A first preferred equation for a correspondingly corrected vagal tone VT is:

$${VT}_{corr} = \frac{median of \{ log [ absolute ( {RR}_{n + 1} - {RR}_{n} ) ] \}}{G} * 100$$

In this case, the median is determined and further corrected. A second preferred equation for the correspondingly corrected vagal tone VT is:

$${VT}_{corr} = \frac{log \vert \frac{1}{N} {\sum }_{i = 0}^{N} [ absolute ( {RR}_{n + 1} - {RR}_{n} ) ] \vert}{G} * 100$$

In this case, instead of the median, the mean is calculated and further corrected.

The coefficient G designates a correction variable. The value of these correction variables is determined by comparing the measured data for the vagal tone with stored data. For this purpose, corresponding measurement values for the vagal tonus are stored in a database, and particularly are linked to as many of the following characteristics as possible: age of the subject, biological sex of the subject, arm length of the subject, body size of the subject, weight of the subject, ethnic origin, regional origin, medication use, drug use, place of residence, eating habits, sleep/waking habits. Furthermore, it has proven to be particularly advantageous if the recorded data for vagal tone—that is, the so-called vagal tone comparison values—are also stored in the database with reference to the time and/or time of day, as well as the season. In the case of a new subject, the data are then read from the database utilizing the corresponding physical characteristics, the current time and/or time of day, as well as further personal conditions for which the given subject has shown suitable and/or similar characteristics. Furthermore, it is advantageous if, in addition, in each case the data which also coincide with regard to the time and/or time of day, as well as the season are used for the current subject.

Because the stored entries are likewise subject to variation, preferably an average value for G is determined for these stored measured values which are suitable in terms of characteristics and/or time and have been selected accordingly. This average value may be the arithmetic mean of the selected vagal tone comparison values, for example. It has been found to be more advantageous if the average value is a median of the selected vagal tone comparison values. It is particularly preferred that the value of G is a percentile value of the selected vagal tone comparison values relative to a limit in %—for example, a 50% or a 90% percentile value—which can be pre-specified and/or will be entered.

G is therefore an average value of a vagal tone determined from stored vagal tone comparison values.

Using the variable coefficient G, the vagal tone VT can be corrected to determine whether it is higher and/or lower than the stored entries, adapted to the respective factors and/or characteristics mentioned. The current vagal tone is therefore normalized over the value G. By multiplying by 100, the output value can be a percentage of G. The corrected VT is then VT_{corr}.

It should be pointed out that the comparison of the comparison data with the current measured data with regard to the time and/or time of day, in particular, has proven to be particularly effective for further increasing the accuracy of the present method.

Like the vagal tone, autonomic tone AT and/or sympathetic tone ST and/or heart rate variability HRV can also be used. In each case, provision is made in particular for these to be corrected in the manner described above.

In the context of the present application, a correspondingly corrected value is preferably formed for all determined values, which is then used subsequently.

The PEP can be determined in different ways from the VT. The following preferred equation has proven to be advantageous; here the corrected VT_{corr} is used directly. Alternatively, a different autonomic tone may be used. In this case, a time value of a pre-ejection period is determined by means of the autonomic tone. Furthermore, the RR interval and/or the heartbeat frequency are preferably incorporated into the equation at the time the measurement is taken.

$${PEP}_{Corr} = [ 0.03 * RR + 88.36 ] - \{ \frac{[ ( 0.03 * RR + 88.36 - ( 0.021 * RR + 89.2 ) ] * {VT}_{Corr}}{100} \}$$

Alternatively, in the above equation, a corrected interval, RR_{corr}, may also be input, which would then take the place of the measured interval and/or the measured time period RR. RR_{corr} would be determined analogously to VT_{corr}, as already explained in detail. In this case, matching comparison data are determined from stored comparison data for RR intervals as well as for the input of the corresponding characteristics and/or the time of the measurement, as described in detail for the coefficient G.

Preferably, a pulse signal of the subject is determined from a peripheral blood vessel by means of a pulse measuring arrangement and transmitted to the circulation parameter determination unit 4. It is particularly preferred that the circulation parameter determination unit 4 also takes into account a determined profile of the pulse signal, in particular a pulse width of a pulse within the pulse signal, for the determination of the at least one blood pressure value.

A pulse arrival time PAT is determined from the heartbeat signal and the pulse signal of the peripheral blood vessel. The pulse arrival time is the time that elapses between a first heartbeat and the occurrence of an associated first pulse signal at the peripheral blood vessel. Different types of determination of this pulse arrival time and/or PAT can be provided. The heartbeat signal is always measured away from the R wave—specifically, away from its appearance, either up to the beginning of the associated pulse wave or up to the maximum slope of the given pulse wave or to the apex of the given pulse wave. The so-called PAT is therefore determined directly from two directly measured values. Furthermore, a mean value for the PAT is preferably determined.

Preferably, in addition to the PAT, a PAT_{corr} is also formed, analogously to the described determination of the VT_{corr}. Therefore, a coefficient over which the PAT is normalized is formed from stored comparison data.

Subsequently, a corrected pulse transit time PTT is determined by deducting the pre-ejection period from the pulse arrival time. PTT stands for pulse transit time. In particular, the following formula has proven to be advantageous:

$$PTT = \{ \frac{1}{N} * {\sum }_{i = 1}^{N} {PAT}_{i} \} - {PEP}_{Corr}$$

Alternatively, instead of the mean value in the above equation, the median PAT may also be used. The PTT can be further corrected.

Furthermore, a value for an arterial length lₐᵣₜ for the peripheral blood vessel of the subject, is determined—in particular, by measuring the corresponding length—and transmitted to the circulation parameter determination unit 4. This length can be determined, for example, using the external size of the subject. In particular, the arterial length lₐᵣₜ is the distance from the valve plane of the heart to the pulse measurement site of the subject's blood vessel, as observed along the relevant blood vessel.

Preferably, the arterial length is taken into account in determining the at least one blood pressure value.

The circulation parameter determination unit 4 determines a pulse wave velocity PWV from the arterial length and the corrected pulse transit time. In this case, as is known:

$$v = \frac{s}{t}$$

Accordingly:

$$PWV = \frac{{l}_{art}}{PTT} and {PWV}_{raw} = \frac{{l}_{art}}{PAT}$$

Furthermore, the circulation parameter determination unit 4 preferably determines the blood pressure value from at least one equation, wherein the equation comprises at least the pulse wave velocity as a variable, as well as a plurality of coefficients. This equation can be a linear system of equations, as well as further quadratic or cubic terms and constants. The following is a first example of corresponding equations for the systolic blood pressure BP_{sys} and the diastolic blood pressure BP_{dia}:

BP_{sys}=S−T*(PWV or PWV_{raw})+H*(PWV or PWV_{raw})²

BP_{dia}=BP_{sys}−{U−V*(PWV or PWV_{raw})+W*(PWV or PWV_{raw})²}

The coefficients S, T, H, U, V and W can be determined in different ways.

According to a first preferred variant, a reference blood pressure is measured directly on the subject in a calibration step and transmitted to the circulation parameter determination unit 4, and the circulation parameter determination unit 4 determines values for the plurality of coefficients by comparing the measured reference blood pressure and the pulse wave velocity. This reference measurement can be determined in a conventional manner by means of a blood pressure measuring cuff, for example. The circulation parameter determination unit 4 then varies values for the respective coefficients until appropriate values are determined. At the same time, a calculation can be made to see how close the approximation comes to the measured values.

The calibration can be improved if a plurality of measurements is carried out for this purpose—for example, at different times of the day and/or in different situations and/or stress conditions. On the basis of this calibration, specific data sets of a plurality of stored data sets can then be assigned to the subject.

According to a second preferred variant, at least one value, in particular a value group, relating to at least one pre-specifiable physical characteristic and/or lifestyle characteristic of the subject is transmitted to the circulation parameter determination unit 4, and the circulation parameter determination unit 4 determines the plurality of coefficients by comparing the input values to stored comparison data sets, wherein coefficients are selected on the basis of a pre-specifiable degree of agreement and/or similarity between the input values and the stored comparison data sets. This variant does not require a reference measurement. However, it is necessary beforehand to measure blood pressure values with a representative quantity and/or group, and to record these together with values, in particular a value group, relating at least one pre-specifiable physical characteristic and/or lifestyle characteristic of the respective measured subject, and thus to form the comparison data sets. This presents no difficulty for a person skilled in the art, since it is merely necessary to carry out a corresponding quantity of measurements.

Preferably, the at least one physical characteristic of the subject is at least one physical characteristic selected from the group: age of the subject, biological sex of the subject, arm length of the subject, body size of the subject, weight of the subject, ethnic origin, regional origin.

Preferably, the at least one lifestyle characteristic of the subject is a lifestyle characteristic selected from the group: medication use, drug use, place of residence, dietary habits, sleep/waking habits.

The next subject needs only to enter the appropriate information and/or transmit it to the circulation parameter determination unit 4, which then selects values for the coefficients based on the best matches.

In particular, in determining the relevant coefficients, the time and/or time of day, as well as the season, are taken into account, since this can further increase the accuracy of the present method.

The coefficients can be determined and/or selected in a simple manner with means of computer-implemented mathematics. The assignment can be made by software, for instance, according to least squares error, or a neural network or methods of artificial intelligence.

As an alternative to the first preferred implementation of the present method described above, a second preferred implementation of the subject method will now be described. This second implementation is more open and/or less specified than the first preferred implementation.

The circulation parameter determination unit 4 determines the systolic blood pressure according to the following preferred relationship, wherein individual terms of this equation can also be omitted:

P_{sys}=a*PWV+b*AT+c*HR+d*EBT+e*PW+f

The circulation parameter determination unit 4 determines the diastolic blood pressure according to the following preferred relationship:

P_{dia}=m*PWV+n*AT+o*HR+p*EBT+q*PW+r

Terms of a higher order than, for example, PWV² and/or PWV³ can also be used for the individual factors of these equations. It has been found that the calculation of these and also of the further presently described values by means of quadratic and/or cubic terms for some groups can produce better results.

Here, AT denotes the “autonomic nervous system tone” or ANS tone. This can be a VT and/or an ST.

HR stands for heart rate.

EBT refers to extremity body temperature—that is, the temperature of am extremity of the subject. Therefore, according to a particularly preferred embodiment, a body temperature is recorded at a peripheral body part of the subject by means of a temperature measuring device, and transmitted to the circulation parameter determination unit 4, and the circulation parameter determination unit 4 takes into account a determined peripheral body temperature in determining the at least one blood pressure value.

PW stands for the “pulse width”—that is to say, the pulse width of an optionally determined peripheral pulse signal. Therefore, according to a particularly preferred embodiment, a pulse signal of the subject is determined by means of a pulse measuring arrangement 3 on a peripheral blood vessel and transmitted to the circulation parameter determination unit 4, and the circulation parameter determination unit 4 takes into account a determined profile of the pulse signal, in particular a pulse width of a pulse within the pulse signal, in the determination of the at least one blood pressure value.

PWV denotes the already described pulse wave velocity.

The equations of the second implementation show how further parameters and/or variables can be included in the calculation of the blood pressure.

The coefficients given in the aforementioned relationships can be determined by means of experiments and/or calibration, as already described for the first implementation.

Furthermore, at least one value for electrical skin resistance, skin temperature, tissue conductivity, tissue capacity, is preferably determined and taken into account in the determination of the at least one blood pressure value. Since it has been shown that these factors can also influence blood pressure and/or autonomic tone—for instance, vagal tone or sympathetic tone—and/or an autonomic rhythm amplitude such as vagal rhythm or sympathetic tone, the measurement can be further improved by taking these into consideration.

Further factors which can be taken into consideration in the present case are the pulse/respiratory quotient, the respiratory rhythm, and the pulse respiratory coupling. For the calculation of the pulse/respiratory quotient, the heart rate—for example, from an ECG—is used. The respiratory rate required for the calculation is calculated, for example, from the modulation of the heartbeat rate by respiration—that is to say, the so-called respiratory sinus arrhythmia.

Preferably, all parameters are corrected by means of comparison data and with reference to time, time of day and/or season, as described in detail with reference to the VT.

In the further described method for determining at least one circulation parameter of a subject, a heartbeat signal of a subject, in particular an ECG or one of the further methods named above, is likewise determined by means of a heartbeat measuring arrangement 2 and transmitted to a circulation parameter determination unit 4. The circulation parameter determination unit 4 determines from the profiles of the heartbeat signal at least one first circulation parameter intermediate value.

Unless otherwise stated, components and/or variables of the two described methods bearing the same reference correspond to the same components and/or variables. In the interpretation of the terms, therefore, the explanations for one method apply equally to the other method.

Preferably, the circulation parameters determined according to the invention are values of at least one autonomic tone. Since determining at least one blood pressure value of the subject from these values is also claimed, a combination of individual or all method steps of the two methods is provided.

Very accurate circuit parameter values can be determined by means of the present method.

At least one value, in particular one value group, relating to at least one pre-specifiable physical characteristic and/or lifestyle characteristic of the subject is transmitted to the circulation parameter determination unit 4. This is done by entering the appropriate information via an interface.

Preferably, the at least one physical characteristic of the subject is at least one physical characteristic selected from the group: age of the subject, biological sex of the subject, arm length of the subject, body size of the subject, weight of the subject, ethnic origin, regional origin. Furthermore, the at least one lifestyle characteristic of the subject is preferably a lifestyle characteristic selected from the group: medication use, drug use, place of residence, eating habits, sleep/waking habits. All of these factors may affect the determination of the circulation parameter value, but need not necessarily.

As a further value, a current time or time of day or season is preferably taken into account when determining the circulation parameter calculation value. Data sets corresponding to different times, times of the day and/or seasons are accordingly stored in the comparison data sets. It has been shown that one and the same measured value of a subject has a different meaning depending on what time of day it is determined. For example, one and the same blood pressure value at midday can be classified as harmless—but in the evening as problematic or even dangerous.

Storing time-based and/or season-based reference data is therefore advantageous because the relationships between the parameters and thus the coefficients over the course of the day and/or year can change; as such, a time-based correction can be made.

The circulation parameter determination unit 4 determines a circulation parameter calculation value from the at least one input value, in particular the value group.

For the determination of the circulation parameter computed value, each of the entered values for physical characteristics and/or lifestyle characteristics is preferably multiplied by a coefficient assigned to the respective physical characteristic and/or lifestyle characteristic.

In this case, the circulation parameter computed value is determined taking into account a large number of comparison data sets which are created from reference measurements and/or have been previously created. The coefficients for the individual physical characteristics and/or lifestyle characteristics are preferably determined by comparing the input values with the values and/or relationships stored in the plurality of comparison data sets. The coefficients for the individual physical characteristics and/or lifestyle characteristics are preferably determined by means of a neural network from the plurality of comparison data sets.

The circulation parameter determination unit 4 determines a circulation parameter output value from the circulating parameter intermediate value, the circulation parameter computed value and also a pre-definable deviation width, and outputs this circulation parameter output value.

Subsequently, a value for the systolic blood pressure and a value for the diastolic blood pressure can each be determined and output from the circulation parameter output value, wherein the steps described above for the determination of a blood pressure value are to be used, wherein autonomic tone is determined according to the present method.

For the determination of the blood pressure, a pulse signal of the test subject is further preferably determined by means of the pulse measuring arrangement 3 at a peripheral blood vessel and transmitted to the circulation parameter determination unit 4, and the circulation parameter determination unit 4 takes into account detected profiles of the peripheral pulse signal in determining the first circulation parameter value.

Furthermore, the vitality of a person can also be monitored in this way and/or a machine can be controlled or an action can be initiated on the basis of a blood pressure value or a value of another circulation parameter. The determined blood pressure is preferably compared to at least one comparison criterion, and if the at least one comparison criterion is fulfilled, a control signal and/or a warning signal is output.

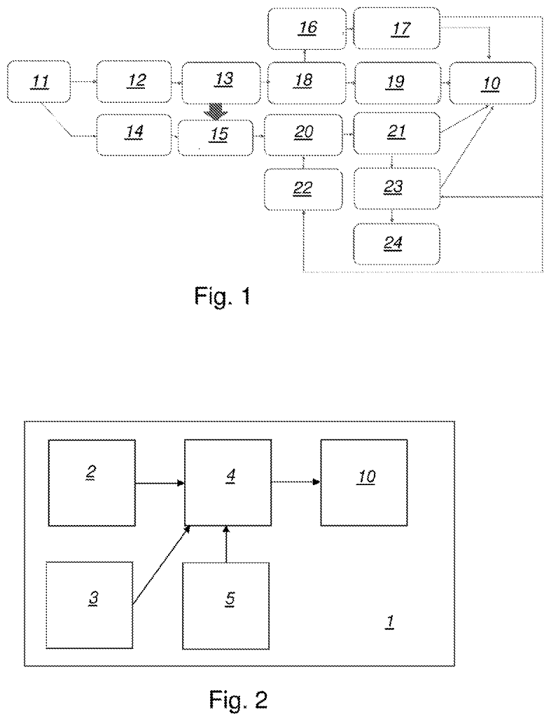

FIG. 1 shows a block diagram of a preferred method sequence for determining a circulation parameter, in particular a blood pressure of a subject. In the diagram, reference number 11 designates the human or animal subject. Of course, the present method is limited to animals with a circulation system.

Reference number 12 denotes the recording of the heartbeat signal, and reference number 13 denotes the detection of the R-wave within the heartbeat signal. In block 18, the time between two R peaks is determined, and in block 16 the AT is determined. In block 19, the heart rate is determined. In block 17, the optional but preferred comparison is made with the stored comparison data sets.

Reference number 10 denotes a display.

Reference number 14 denotes the recording of the pulse wave at the peripheral vessel. At reference number 15, starting from an expected occurrence time, starting from the detected R wave, a time window is placed over the pulse wave signal. Reference number 20 denotes the detection of the pulse wave within the monitored time window of the pulse wave signal.

In block 21, the PWV is determined. In block 23, the blood pressure values are determined. This determination can be made by means of the determined AT values.

Reference number 22 denotes the correction of the PEP.

In block 24, the control of a machine is provided.

According to a further preferred method, a stimulus and/or a respiratory challenge can be generated and output according to a profile of the blood pressure and/or the circulation parameter output value. This allows the blood pressure curve to be used for biofeedback.

A change in the blood pressure and/or the circulation parameter output value is preferably determined in response to the output stimulus and/or the respiratory request, and the change is taken into account in the determination of subsequent time points for outputting the stimulus and/or the respiratory challenge. It has been shown that the specification of stimuli and/or respiratory challenges described herein can enable achieving a targeted influence on the blood pressure profile of a subject. As a result, a particular condition of a subject can be specifically produced which has a physical as well as a mental effect the same. In this way, the well-being and performance of a subject can be sustainably and easily increased. This can improve the health of a subject.

In addition, a device 1 for determining at least one circulation parameter of a subject is provided, the device comprising a heartbeat measuring arrangement 2, a pulse measuring arrangement 3, and a circulation parameter determination unit 4, wherein the device 1 comprises a data memory 5 having a plurality of comparison data sets, the same comprising relationships between values for physical characteristics and/or lifestyle characteristics of the subject and circulation parameter values of the subject, and the circulation parameter determination unit 4 is designed to carry out a method according to the invention. FIG. 2 shows a block diagram of a corresponding device.

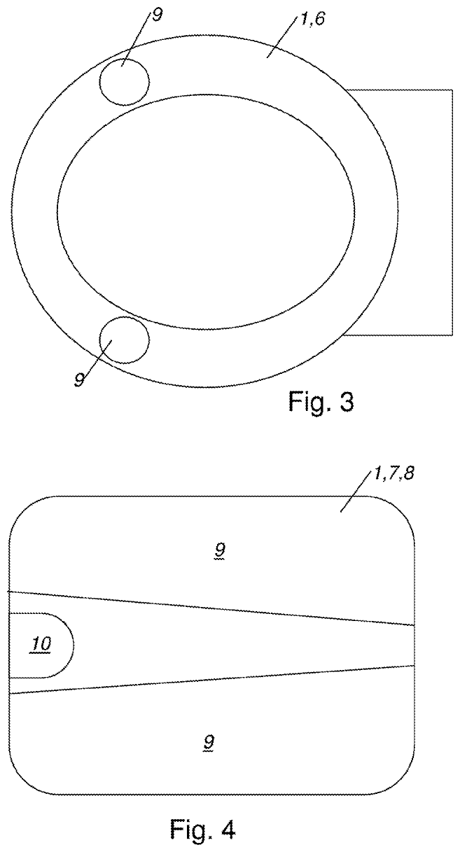

According to a first preferred embodiment, at least contact regions 9 of the heartbeat measuring arrangement 2 and/or the pulse measuring arrangement 3 are arranged in a seat surface, in particular a toilet seat surface 6. FIG. 3 shows a schematic representation of a corresponding toilet seat. In further preferred embodiments, the seat surface is part of a bathtub and/or a work and/or office chair. Likewise, a lying surface, in particular a therapy couch or a bed can be designed accordingly.

According to a second preferred embodiment, at least contact regions 9 of the heartbeat measuring arrangement 2 and/or the pulse measuring arrangement 3 are arranged in a standing surface 7, in particular the standing surface 8 of a scale. FIG. 4 shows a schematic representation of a corresponding scales. In further preferred embodiments, the standing surface is designed as part of a shower tray.

According to a third preferred embodiment, at least contact areas 9 of the heartbeat measuring arrangement 2 and/or the pulse measuring arrangement 3 are arranged in a control element and/or operating element, in particular a steering wheel, a handlebar, a control wheel and/or a joystick, or a machine, in particular a land- and/or air- and/or watercraft.

In the method described herein, it is not absolutely necessary that each determined parameter and/or all available parameters are incorporated in the determination and/or calculation of a value. It has proven to be particularly advantageous to determine certain values several times using different parameters and/or a combination of parameters, and in each case to generate an error probability for these determinations, then to determine a final output value to determine from a comparison and/or merging of the differently determined values.

1-16. (canceled)

17. A method for determining at least one blood pressure value of a test subject, comprising:
determining a heartbeat signal of a subject by means of a heartbeat measuring arrangement;
transmitting the heartbeat signal to a circulation parameter determination unit;
determining, by the circulation parameter determination unit, at least one value of an autonomic tone from a determined time profile of the heartbeat signal;
determining, by the circulation parameter determination unit, the at least one blood pressure value taking into account the at least one value of the autonomic tone; and
outputting the determined blood pressure value.

18. The method according to claim 17, wherein the heartbeat signal is an ECG.

19. The method according to claim 17, wherein the autonomic tone comprises at least one of a vagal tone, a heart rate variability, a sympathetic tone, and an autonomic quotient.

20. The method according to claim 17, wherein the value of the autonomic tone is normalized using stored comparison data sets before it is taken into account in the determination of the blood pressure value.

21. The method according to claim 17, wherein a time value of a pre-ejection period is determined by the circulation parameter determination unit by means of the autonomic tone, and in that the circulation parameter determination unit takes the pre-ejection period into account in the determination of the at least one blood pressure value.

22. The method according to claim 17, wherein, by means of a pulse measuring arrangement, a pulse signal of the subject is determined on a peripheral blood vessel and transmitted to the circulation parameter determination unit, and in that the circulation parameter determination unit takes into account a determined profile of the pulse signal, in particular a pulse width of a pulse within the pulse signal, in the determination of the at least one blood pressure value.

23. The method according to claim 22, wherein a pulse arrival time is determined from the heartbeat signal and the pulse signal on the peripheral blood vessel, wherein the pulse arrival time is the time which elapses between a first heartbeat and the appearance of an associated first pulse signal in the peripheral blood vessel.

24. The method according to claim 23, wherein a corrected pulse transit time is determined by subtracting the pre-ejection period from the pulse arrival time.

25. The method according to claim 22, wherein a value is determined for an arterial length of the peripheral blood vessel of the subject and is transmitted to the circulation parameter determination unit, and in that the circulation parameter determination unit takes into account the arterial length in the determination of the at least one blood pressure value.

26. The method according to claim 25, wherein the value is determined for the arterial length of the peripheral blood vessel of the subject by measuring a corresponding length.

27. The method according to claim 25, wherein a pulse wave velocity is determined by the circulation parameter determination unit from the arterial length and the corrected pulse transit time.

28. The method according to claim 27, wherein the circulation parameter determination unit determines the blood pressure value from at least one equation, which equation has at least the pulse wave velocity as a variable, as well as a plurality of coefficients.

29. The method according to claim 28, wherein, in a calibration step a reference blood pressure is measured directly on the subject and transmitted to the circulation parameter determination unit, and in that the circulation parameter determination unit determines values for the plurality of coefficients by comparing the measured reference blood pressure and the pulse wave velocity.

30. The method according to claim 28, wherein at least one value relating to at least one predetermined physical characteristic and/or lifestyle characteristic of the subject is transmitted to the circulation parameter determination unit, and in that the circulation parameter determination unit determines the plurality of coefficients by comparing the input values to stored comparison data sets, wherein coefficients are selected based on at least one of a pre-specifiable measure of agreement and a similarity between the input values and the stored comparison data sets.

31. The method according to claim 30, wherein the at least one physical characteristic of the subject is at least one physical characteristic selected from the group: age of the subject, biological sex of the subject, arm length of the subject, body size of the subject, weight of the subject, ethnic origin, and regional origin.

32. The method according to claim 30, wherein the at least one lifestyle characteristic of the subject is a lifestyle selected from the group: medication use, drug use, place of residence, eating habits, and sleep/waking habits.

33. The method according to claim 30, wherein one of a current time, a time of day, and a season is taken into account in determining the blood pressure value.

34. The method according to claim 33, wherein each of the comparison data sets includes the one of a current time, a time of day, and a season at which they were created.

35. The method according to claim 30, wherein in each case a first value for at least one of the systolic blood pressure and a first blood pressure amplitude and a second value for at least one of the diastolic blood pressure and a second blood pressure amplitude is determined and output.

36. A device for determining at least one circulation parameter of a subject, the device comprising:
a heartbeat measuring arrangement;
a pulse measuring arrangement; and
a circulation parameter determination unit,
wherein the device is designed to carry out a method according to claim 17.
